(19) United States
(12) Patent Application Publication
(10) Pub. No.: US 2007/0285651 A1
(43) Pub. Date: Dec. 13, 2007

(54) Methods and devices for measuring sample coagulation

(76) Inventor: Thomas L. Cantor, El Cajon, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/789,493

(22) Filed: Apr. 24, 2007

Related U.S. Application Data
(60) Provisional application No. 60/794,869, filed on Apr. 24, 2006.

(51) Int. Cl. G01N 33/48; G01N 21/00

(52) U.S. Cl. 356/39; 356/434

(57) ABSTRACT

The present invention provides devices and methods for measuring coagulation time. The device comprises a slide coupled to a sample carrier capable of sample flow from one end of the carrier to another end in a continuous back and forth motion until sample coagulates thereby preventing sample flow; a rocker or shaker means for moving sample inside the sample carrier; a light detector assembly for detecting light transmitted through said sample carrier; and a display means for indicating coagulation time corresponding to substantially no change in the amount of light transmitted as indicative of sample coagulation.

### CROSS-REFERENCE TO RELATED APPLICATIONS

This application claims priority under 35 U.S.C. § 119(e) from provisional patent application No. 60/794,869, filed Apr. 24, 2006. The content of the above patent application is incorporated by reference herein in its entirety.

### BACKGROUND OF THE INVENTION

1. Technical Field

The invention relates to apparatus and methods for measuring coagulation time.

2. Background Art

Anticoagulants or blood thinners are a common treatment for preventing blood clots and strokes. A large number of patients take anticoagulants, but their reactions to the treatment must be tested regularly to prevent bleeding or hemorrhage, and to assure that they have sufficient effective anti-coagulation therapy. Patients who frequently monitored their own treatment and adjusted their dose have been found to suffer fewer blood clots and deaths than people tested on a less frequent basis by medical professionals.

Self-monitoring is important because blood-thinners can interact with antibiotics and alcohol, and are affected by certain types of food. Monitoring is done almost daily in the early days of treatment, and can be reduced over time once coagulation times have been stabilized. Generally, self-monitoring involves taking a blood sample from a pin prick (i.e., lancet puncture), and using it into a home coagulation testing kit. The machine gives a reading that shows if the patient is in a safe range (i.e., target coagulation time range or TCR) and not at risk of uncontrolled bleeding or having a dangerous blood clot. If it is found that the patient is not in the TCR then the anticoagulant dosage may be easily adjusted. Although various devices for determining and measuring coagulation times are known, there remains a need for a coagulation measuring device that is simple, accurate and easy to operate for home use.

### DISCLOSURE OF THE INVENTION

The present invention provides an apparatus and method for operating the apparatus for measuring coagulation time that is simple, accurate and easy to operate.

In one embodiment, the present invention provides a device comprising a slide coupled to a sample carrier capable of sample flow from one end of the carrier to another end in a continuous back and forth motion until sample coagulates thereby preventing sample flow; a rocker or shaker means for moving sample inside the sample carrier; a light detector assembly for detecting light transmitted through said sample or sample carrier; and a display means for indicating coagulation time corresponding to substantially no change in the amount of light transmitted as indicative of sample coagulation. The sample may be blood from a mammalian subject, and may be blood from a diabetic patient.

Various sample carriers, rockers, shaker means, light detector means, and display means may be used to practice the apparatus and methods of the invention. In some examples, the sample carrier is a capillary tube such as a hematocrit tube. The shaker means may be a mechanical or a manual shaker. The display means may be a digital display means. The light detector may comprise a laser light source.

In some embodiments, the device of the present invention may further comprise a housing. For example, the housing may comprise a hinged structure for opening and closing said housing. In some examples, the hinged structure is configured such that closing the housing operates to turn on the light source. The light source then shines a beam of light through the slide and through the end of the sample carrier opposite to where the sample is placed. In other examples, the hinged structure is configured such that closing the housing operates to turn on a timer for measuring coagulation time. In yet other examples, the hinged structure is configured such that closing the housing operates to turn on the shaker means.

In yet other embodiments, the light detector assembly comprises a light source and a photocell. The light beam may be transmitted from the light source through the sample carrier onto the photocell, which produces an output corresponding to the amount of light transmitted. In some examples, an output corresponding to the formation of sample coagulation is produced when the range of variation in the amount of light transmitted through the sample carrier is within 0-10%. In other examples, the range of variation is 0 to 5%. In yet other examples, the range of variation is 0 to 1%.

The present invention also provides a method for determining coagulation time using the device as described above. In one embodiment, the present invention provides a method for measuring the coagulation time of a blood sample using the device as described above. In one embodiment, the present invention provides a method for determining coagulation time, comprising: a) applying a blood sample to a target location on a slide having disposed onto a capillary tube; b) initiating a timing cycle when sample inside capillary tube is moved by means of a shaker from one end of the tube to another end in a continuous back and forth motion until sample coagulates thereby preventing sample flow, and a light source is turned on for transmitting light through the tube; and c) determining coagulation time when substantially no change in the amount of light transmitted is detected.

The above summary of the present invention is not intended to describe each disclosed embodiment or every implementation of the present invention. Various changes and modifications to the disclosed embodiments will be apparent to those skilled in the art.

### BRIEF DESCRIPTION OF THE DRAWINGS

FIG. 1 illustrates one embodiment of the apparatus of the present invention.

### MODES OF CARRYING OUT THE INVENTION

The present invention provides an apparatus and method for operating the apparatus for measuring coagulation time that is simple, accurate and easy to operate. The apparatus of the present invention may be used to measure the coagulation time using a variety of samples where coagulation time measurement is important. The apparatus of the present invention does not require transport to a laboratory for analysis, and avoids the risk of deteriorating clotting factors that may lead to inaccurate results.

The apparatus of the present invention may be used for bedside professional use and for home use, and is particularly useful for self-monitoring of patients taking anticoagulants. For example, the coagulation measuring device may be used by dialysis patients to measure blood coagulation time, and self-monitor the dosage of anticoagulant taken before and after dialysis. The amount of coagulation time measured may be compared against a range of coagulation times considered safe and normal. Thus, patients having a coagulation time measured outside the safe and normal range may adjust the dosage of anticoagulant taken, or seek professional care thereby preventing the risk of bleeding or having a blood clot. In like manner, the device may be used by professionals who require a rapid, accurate and easy to perform method for determining coagulation times. For example, the device may be used by a dialysis nurse or technician managing the coagulation status of an end stage renal disease (ESRD) patient.

In one embodiment, the present invention provides a device comprising a slide coupled to a sample carrier capable of sample flow from one end of the carrier to another end in a continuous back and forth motion until sample coagulates thereby preventing sample flow; a rocker or shaker means for moving sample inside the sample carrier; a light detector assembly for detecting light transmitted through said sample or sample carrier; and a display means for indicating coagulation time corresponding to substantially no change in the amount of light transmitted as indicative of sample coagulation.

The coagulation time may be measured starting from the initial flow of the uncoagulated sample through continuous flow of the uncoagulated sample from one end of the sample carrier to the other end of the carrier, until there is no more flow of coagulated sample. The light detector assembly detects the presence or absence of flow of coagulated sample. In one embodiment, the light detector assembly detects no transmitted light when coagulated sample blocks the light source. Alternatively, the light detector assembly detected transmitted unblocked light when sample coagulation (corresponding to cessation of back and forth flow of sample in the tube) occurred away from the light path. When there is substantially no change in the amount of light transmitted following back and forth rotation of the platform, the light detector produces an output corresponding to the formation of sample coagulation, and the display means displays the sample coagulation time. In particular examples, the display means provides a digital display of coagulation time.

In one embodiment, the light detector assembly comprises a light source and a photocell, wherein a light beam is transmitted from the light source through the sample carrier onto the photocell, which produces an output corresponding to the amount of light transmitted through the sample carrier. In one example, an output corresponding to the formation of sample coagulation is produced when the range of variation in the amount of light transmitted through the sample carrier is within 0-10%. In other examples, the range of variation is within 0-5%. In yet other examples, the range of variation is within 0-1%.

The sample coagulation measuring device of the present invention may further comprise a control switch means coupled to the light detector assembly, which operates to turn the shaker means in response to the output obtained from the light detector assembly corresponding to the formation of sample coagulation. The light detector assembly, display means and the control switch means may be configured in various ways as known to those skilled in the art. (See e.g., U.S. Pat. Nos. 3,593,568; 4,640,896; 4,964,728; and 5,344,754, each of which is incorporated herein by reference in its entirety). The apparatus of the present invention may be battery operated, or powered by a non self contained electrical source.

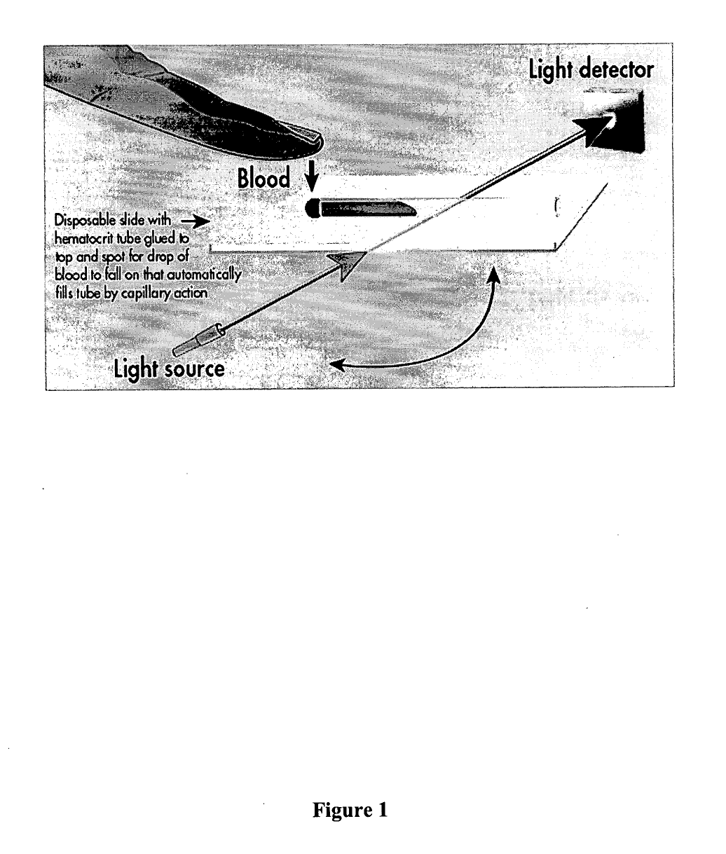

FIG. 1 illustrates one embodiment of the coagulation measuring device of the present invention, comprising a tube 1 disposed on slide 2, a light source and a light detector assembly. In one embodiment, the tube is a capillary tube or a hematocrit tube that is supported on the slide with an adhesive. A drop of blood, which may be obtained from the finger of a patient using a lancet, is placed upstream on one end of slide 2. A rocker or shaker means such as a rocking platform moves the slide and the tube disposed on the slide, such that sample flows back and forth within the tube.

The device may be placed within a housing, which may comprise a hinged structure for opening or closing the housing. The hinged structure may be configured such that closing the housing operates to turn on the light source that shines a beam of light through the slide and through the end of the tube opposite to where the blood sample was placed, and to simultaneously start the back and forth rocker on which the slide is resting. A timer starts when the hinged structure is closed and the slide unit starts to rock back and forth. When the detector detects that there is no more flow back and forth of the sample, the timer is stopped and displayed on a screen as the clotting time.

The shaker means may be any suitable shaker that causes the sample to move back and forth inside the sample carrier, and may be a mechanical or a manual shaker. The following assays using a manual shaker were run to demonstrate the apparatus and methods of the invention. Each tube in each assay was gently rocked back and forth by hand, following the movement of a rocking chair, at an angle of about 30 degrees. In assay 1, the tube is filled with a drop of blood obtained from the arms of a subject, and the coagulation time was measured at 4 minutes and 54 seconds. In assay 2, the tube is filled by dipping the tube into a vaccutainer containing blood obtained from the same donor as in assay 1, and the coagulation time was measured at 5 minutes and 42 seconds. In assay 3, the tube is filled with a drop of blood obtained from the finger tip of a second donor, and the coagulation time is measured at 4 minutes and 51 seconds.

The results show that assay 1 and assay 3 had similar coagulation times, varying only by a few seconds. On the other hand, assay 2 had a longer coagulation time, differing from assays 1 and 3 by almost 60 seconds. The difference in coagulation times may be due to a sample overload in assay 2 when the tube was dipped into a vaccutainer, resulting in a greater volume of sample. The degree of movement may also contribute to the variation in coagulation times. For evaluation of reproducibility and CV determination, various shaker means known in the art may be tested.

It is understood that the foregoing detailed description and accompanying examples are merely illustrative, and are not to be taken as limitations upon the scope of the invention. Various changes and modifications to the disclosed embodiments will be apparent to those skilled in the art. U.S. patents and publications referenced herein are incorporated by reference.

1. A device comprising a slide coupled to a sample carrier capable of sample flow from one end of the carrier to another end in a continuous back and forth motion until sample coagulates thereby preventing sample flow; a rocker or shaker means for moving sample inside the sample carrier; a light detector assembly for detecting light transmitted through said sample or sample carrier; and a display means for indicating coagulation time corresponding to substantially no change in the amount of light transmitted as indicative of sample coagulation.

2. The device of claim 1, wherein the sample is blood.

3. The device of claim 1, wherein the sample carrier is a capillary tube.

4. The device of claim 1, wherein the shaker means is a mechanical or a manual shaker.

5. The device of claim 1, wherein the display means is a digital display means.

6. The device of claim 1, further comprising a housing.

7. The device of claim 6, wherein said housing comprises a hinged structure for opening and closing said housing.

8. The device of claim 7, wherein said hinged structure is configured such that closing the housing operates to turn on the light source.

9. The device of claim 8, wherein said light source shines a beam of light through the slide and through the end of the sample carrier opposite to where the sample is placed.

10. The device of claim 1, wherein the light source is a laser light.

11. The device of claim 7, wherein said hinged structure is configured such that closing the housing operates to turn on a timer for measuring coagulation time.

12. The device of claim 7, wherein said hinged structure is configured such that closing the housing operates to turn on the shaker means.

13. The device of claim 1, wherein the light detector assembly comprises a light source and a photocell.

14. The device of claim 13, wherein a light beam is transmitted from the light source through the sample carrier onto the photocell, which produces an output corresponding to the amount of light transmitted.

15. The device of claim 14, wherein an output corresponding to the formation of sample coagulation is produced when the range of variation in the amount of light transmitted through the sample carrier is within 0-10%.

16. The device of claim 15, wherein the range of variation is 0 to 5%.

17. The device of claim 15, wherein the range of variation is 0 to 1%.

18. A method for determining coagulation time using the device of claim 1.

19. The method of claim 18, for measuring the coagulation time of a blood sample.

20. A method for determining coagulation time, comprising:
a) applying a blood sample to a target location on a slide having disposed onto a capillary tube;
b) initiating a timing cycle when sample inside capillary tube is moved by means of a shaker from one end of the tube to another end in a continuous back and forth motion until sample coagulates thereby preventing sample flow, and a light source is turned on for transmitting light through the tube; and
c) determining coagulation time when substantially no change in the amount of light transmitted is detected.
